(19) United States
(12) Patent Application Publication
(10) Pub. No.: US 2022/0072210 A1
(43) Pub. Date: Mar. 10, 2022

(54) TREATMENT OF VIRAL INFECTION BY APHERESIS

(71) Applicant: ELIAZ THERAPEUTICS, INC., Sebastopol, CA (US)

(72) Inventor: Isaac ELIAZ, Santa Rosa, CA (US)

(73) Assignee: ELIAZ THERAPEUTICS, INC., Sebastopol, CA (US)

(21) Appl. No.: 17/017,502

(22) Filed: Sep. 10, 2020

(51) Int. Cl. A61M 1/34; A61M 1/36; A61K 31/732; C07K 16/10; A61K 35/16

(52) U.S. Cl. CPC A61M 1/3496; A61M 1/362; A61K 31/732; A61M 2202/0415; A61K 35/16; A61M 1/3618; C07K 16/10

(57) ABSTRACT

The invention is directed to the treatment of COVID-19 patients by withdrawing SARS-CoV-2 viral particles from the patient's circulation by apheresis using a binding agent in either a fixed bed, or in a form easily removed, such as by being complexed with magnetic particles. The reduction in viral particles may be combined by reduction of active gal-3 levels in the patient which may provide further relief of conditions associated with COVID-19 that may include symptoms associated with the cytokine storm that is associated with COVID-19 infection. Both SARS-CoV-2 viral particles and gal-3 may be bound by modified citrus pectin of less than sixty thousand Daltons. The process may be combined with the administration of supportive agents like antivirals, anti-inflammatories, immune based inhibitors, vitamins and modified citrus pectin.

### PRIORITY DATA AND INCORPORATION BY REFERENCE

This application is related to, but does not claim priority from, U.S. Pat. Nos. 8,764,695; 10,213,462; U.S. patent application Ser. No. 15/081,958 (allowed) and U.S. patent application Ser. No. 15/104,302. Each of these U.S. Patents and applications is incorporated herein-by-reference. Taken together, these patents clearly establish the efficacy of plasmapheresis to reduce the level in a mammal such as a human the level an agent that binds to a target also bound by circulating galectin-3 (gal-3 herein).

### BACKGROUND OF THE INVENTION

### Field of the Invention

This invention pertains to treatment of viral infections, which may include infections from MERS, SARS, SARS-CoV-2 and other opportunistic viruses. While this application focuses on SARS-CoV-2 and the disease caused thereby, COVID-19, treatment of other viruses and related viral agents may be affected by the same process. In late 2019, scientists in China first identified the emergence of a new virus. Sequencing of the genetic structure of that virus showed a strong morphological resemblance to other coronaviruses, and related viridae. Particular structural similarities to other coronavirus “spike proteins”—the proteins usually used by the virus to initiate cell penetration has been noted by those attempting to find a mechanism to treat and prevent the disease associated with infection by this coronavirus—COVID-19. See, e.g., Caniglia et al, Biochemistry, Biophysics & Molecular Biology, 2020 (pp. 1-10). Indeed, a structural review of the virus and its spike protein strongly suggests that some of the binding properties of the virus are related to those of gal-3—the structural similarities suggest that in fact the binding properties of the spike protein are not distinct from that of gal-3 itself. Revila et al, Frontiers in Immunology, August, 2020 (pp. 1-6).

This structural similarity is reinforced by the biological and pathological parallels between the virus, its disease, and the activities of properties of Gal-3 in the mammalian, and in particular, human body. Thus, as recounted in Caniglia et al, the pro-inflammatory response that is the signature of COVID-19 infection in the lungs of humans is a response activated by gal-3 (involving the release of both IL-6 and TNF-α. The distribution of gal-3 in healthy humans reflects the distribution of distress in infected humans. The highest gal-3 in healthy individuals is in the lungs, followed by the gastrointestinal tract and then the brain. Remarkably, COVID-19 infected individuals exhibit predominantly symptoms of lung inflammation and fibrosis, followed by gastrointestinal symptoms (diarrhea, nausea and vomiting) followed, in turn, by neurological symptoms (cerebrovascular events, seizures, headaches and impaired consciousness). The striking parallel between gal-3 expression in the body, and COVID-19-induced gal-3 mediated events including inflammation, fibrosis, acute kidney injury and the like have led researchers to call for immediate exploration of gal-3 inhibitors as a possible treatment for COVID-19. Revilla et al—Hyperinflammation and Fibrosis in Severe COVID-19 Pateints:Galectin-3, a Target Molecule to Consider (2020).

The strong relationship between the structure and activity of SARS-CoV-2 spike protein and the structure of gal-3 is echoed in the T cell response of those with COVID-19 who survive that infection. They exhibit a strong CD4 T cell response. A critical domain in the spike protein of β-coronaviridae is nearly identical in morphology to human Gal-3. The spike proteins are critical for the virus' entry into host cells, and its aggressive virulence. Gal-3 inhibition could be a path to a COVID-19 solution by a dual mechanism of reducing the host inflammatory immune response, T-cell activation, and interrupting viral attachment to host cells. A specific protocol for inhibiting gal-3 activity, and in particular, the activation and release mechanisms mediated by gal-3 specific to COVID-19, is not yet established.

Establishing a safe and effective protocol for the administration of agents, be they inhibitors of gal-3 or other possible players in the “cytokine storm” phenomena that overwhelms so many COVID-19 patients, is necessarily a time consuming and painstaking process. While some potential agents, such as belopectin and other modified citrus pectin compositions, have been demonstrated safe (the inventor named herein as well as others. has demonstrated the same with Pectasol-C a proprietary MCP composition) there remains a high threshold to demonstrate effectiveness and suitable dosages for all infected individuals.

While the search for other effective measures must continue—Applicant demonstrated well prior to the current coronavirus pandemic the safe and effective removal of gal-3 from human circulation—in order to address other conditions mediated by gal-3. See, U.S. Pat. No. 8,764,695. Further research has demonstrated that other agents morphologically and biologically similar to gal-3 can be bound and removed in the same fashion. The key to this approach has been “selective withdrawal.” In this method of apheresis, blood or a component thereof such as plasma (when plasma is separated out and treated, the process is often referred to a plasmapheresis) is withdrawn from the body and passed through a module or “column” which selectively binds the target—which may be gal-3 and may be another cytokine storm active agent like TNF Alpha and IL-6. and thereafter returned to the body. A device specifically designed for this purpose is set forth in U.S. patent application Ser. No. 15/104,302, which specifically discloses that the blood is withdrawn from the human patient, optionally separated to provide plasma on the one hand and blood cells on the other, and then passed through at least one but often more than one column device for the selective withdrawal of one or multiple targets in the blood/plasma of the patient. Thus, to amplify the effects of selective withdrawal of gal-3 from the patient in controlling the patients reaction, TNF alpha, IL-6, IL 1B, CRP, and other cytokines which can often be activated by gal-3 in the body, may also be withdrawn in a single pass. See U.S. Pat. No. 10,213,462. Thereafter, the gal-3 reduced plasma or blood is returned to the patient (where plasmapheresis is practiced, the plasma and blood cells are combined prior to return to the patient).

The remarkable morphological and biological similarities between gal-3 and SARS-CoV-2, particularly the spike protein thereof make it possible to take advantage of this well established technology that does not require the administration of any active agent to the infected patient and yet allows for safe, effective and rapid withdrawal of a virus that binds to a known target, like the SARS-CoV-2 viral agent, making treatment of the patient using conventional supportive measures safe and effective.

### SUMMARY OF THE INVENTION

Thus, in a preferred but simplified embodiment, the COVID-19 patient's blood or plasma is withdrawn and passed through a column device in which it is exposed to a target that the virus will bind to. One such target is modified citrus pectin up to sixty thousand Daltons molecular weight. The SARS-CoV-2 virus, through the protein spike feature, binds to (or is bound by) the modified citrus pectin (MCP) in the very same fashion that MCP binds gal-3, as shown in U.S. patent application Ser. No. 15/081,978. It is of course true that other agents that SARS-CoV-2 will bind tightly with, and can be immobilized in a column, in particular, antibodies that bind to the virus, can be used as well. Multiple antibodies to the virus that causes COVID-19 have been developed in the race to establish protocols for testing as well as treatment for the disease bind to the virus with sufficient affinity to reduce the level of virus in the blood of the patient after a single passage through the apheresis device. Any of these, as well as a cocktail of agents, advantageously, can be used, instead of, or together with, MCP to bind sufficient viral bodies to reduce a patient's viral load to the point where conventional supportive treatments as well as the patient's own natural resources can effectively carry the patient forward to recovery. Typical antibodies include the IgG antibodies employed in current COVID-19 test kits, such as Test 164055 available from Labcorp. Antibodies CV1 and CV30, specifically targeting the SARS-CoV-2 spike protein are widely available, as are many others. Among available antibodies, many can be identified. Representative suitable antibodies include Antibody TB201-2 from Twist Biosciences (S. San Francisco)—High affinity Anti-S1 domain (of SARS-CoV-2 Spike protein) VHH Single Domain Antibody; Antibody Clone 2C1 (Catalog #MABX8405) from MilleporeSigma (Burlington, Mass.)—Humanized mAb against SARS-CoV-2 Spike protein; and Antibody Clone D005 (Catalog #NBP2-90989) from Novus Biologicals (Centennial, Colo.)—Human recombinant mAb (IgG1) against the RBD domain of the Spike protein from SARS-CoV-2 and SARS-CoV-1. See also, generally, https://absoluteantibody.com/anti-coronavirus-antibodies.

In a preferred embodiment, the plasma or blood is passed through one or more columns where the plasma is exposed to MCP or other binding agents such as antibodies to SARS-CoV-2 and antibodies/affinity ligands to gal-3. As observed above, the morphological and biologically active characteristics of both the viral particles and gal-3 are so close that experts have indicated that the viral spike protein and gal-3 are derived from a common ancestor at some point in history. They will both bind to the same target (e.g., MCP) or can be bound by different targets (e.g., antibodies fashioned for each to achieve selective withdrawal of the virus, and gal-3), in a single passage through the plasmapheresis device. This advance tremendously simplifies matters for this invention. Of course, other targets may be withdrawn, where appropriate, such as TNF Alpha and IL-6.

Agents may be introduced while the blood is withdrawn from the body, particularly the administration of support compositions including ones established with some efficacy for COVID-19 such as antivirals (examples being Remdesivir, favipiravir and merimepodib); anti-inflammatories (Corticosteroids such as Dexamethasone, prednisone, methylprednisolone or hydrocortisone, and Nonsteroidal anti-inflammatory drugs (NSAIDs) such as Ibuprofen, Aspirin, Naproxen as well as Anti-inflammatory botanicals- like Padma Basic formula, quercetin, curcumin); immune based inhibitors (such as IL-6 inhibitors, TNF Alpha inhibitors); vitamin based therapy (such as vitamins C and D). Applicant also notes the MCP (or other gal-3 inhibitors) may play a dual role, serving as the binding agent in the apheresis column(s) and administered orally, iv, in inhalation, or intranasally and intrabuccal as a gal-3 blocker. Other regimens known to those of skill in the art, such as protocols for the protection and treatment of, e.g., kidney patients at risk maybe appropriate. See, e.g., Gabarre et al, Intensive Care Med, 2020 and Shao et al, Pharmacological Research 161 (2020) (pp. 1-14) both of which are incorporated herein-by-reference for their discussion of conventional supportive treatments for COVID-19 patients with underlying kidney issues. These references discuss, for example, the conventional use of diuretics, acetylcysteine and sodium bicarbonate, which may be administered in conjunction with this invention. They will find greater efficacy if administered in conjunction with the selective withdrawal of SARS-CoV-2 and/or gal-3, as the reduction in gal-3 can reduce the need to weather the “cytokine storm.”

### DETAILED DESCRIPTION OF THE INVENTION

Galectins are a family of lectins (sugar binding proteins) that are characterized by having at least one carbohydrate recognition domain (CRD) with an affinity for beta-galactosides. These proteins were recognized as a family only recently, but are found throughout the animal kingdom, and are found in mammals, birds, amphibians, fish, sponges, nematodes and even fungi. This application focuses on gal-3 in mammals, and in particular, humans.

Galectins mediate and modulate a wide variety of intracellular and extracellular functions, and thus are both expressed within the cell and frequently targeted to a specific cytosolic site, and secreted from the cell, for distribution extra-cellularly, as a component of human plasma. Among the many functions that are mediated by extracellular gal-3 are inflammation, fibrosis formation, cell adhesion, cell proliferation and metastatic formation (cancer) and immunosuppression.

Quite clearly, mediation of inflammatory and fibrotic pathways makes galectins critical elements of a wide variety of disease, injury and trauma related phenomena. In many cases, the presence of unwanted concentrations of galectins can aggravate a disease condition or trauma situation, or interfere with attempts to treat diseases, such as cancer, kidney disease (both acute kidney injury-AK1, and chronic kidney disease of all etiologies) or congestive heart failure. A wide variety of conditions in humans, ranging from problems in conceiving to asthma to chronic heart failure to cancer to viral infection to stroke and beyond are mediated or aggravated by higher than normal concentrations of galectins. Thus, among other galectins, gal-3 is particularly prominent in fibrosis, inflammation and cell proliferation and the like. Indeed, the inventor has pioneered efforts to address conditions like inflammation and fibrosis by administration of gal-3 binders like MCP. See, for instance, U.S. Pat. No. 9,649,329.

The discussion herein is focused on the selective withdrawal of SARS-CoV-2 and may include gal-3, since they can be specifically addressed by the same or similar binding elements and a reduction in both may work synergistic benefit in the patient. Gal-3 mediates a large number or events in the cytokine storm that overwhelms patients. More specifically, this invention focuses on the removal of SARS-CoV-2 from mammalian, particularly human, plasma, but also provides for reduction in Gal-3 levels where desired. Gal-3 has been shown to be involved in a large number of biological processes, many of which are related to disease states of various kinds that may be present in individuals infected with COVID-19. Removal of large amounts of gal-3 from circulation may therefore improve existing medical treatments, suppress and/or reduce inflammation and fibrosis resulting from COVID-19 infection, and make it possible to intervene in various disease states not otherwise easily treated. It should be borne in mind that other viral infections (e.g., MERS, SARS, H1N1, etc.) can be treated in the same fashion—antibodies or proteins that they selectively bind to may be used in the same fashion that MCP or antibody binding agents can be (antibody as used herein is a generic term that includes antibodies, antibody fragments, complexed binding elements and the like).

An antibody fragment is defined herein broadly, and generally means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of a Fab (Fd) fragment, which consists of the VH and CH1 domains; (iv) a variable fragment (Fv) fragment, which consists of the VL and VH domains of a single arm of an antibody, (v) a domain antibody (dAb) fragment, which comprises a single variable domain; (vi) an isolated complementarity determining region (CDR); (vii) a Single Chain Fv Fragment; (viii) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites; and (ix) a linear antibody, which comprises a pair of tandem Fv segments (VH-CH1-VH-CH1) which, together with complementarity light chain polypeptides, form a pair of antigen binding regions; and (x) other non-full length portions of heavy and/or light chains, or mutants, variants, or derivatives thereof, alone or in any combination. In addition, antibody CDRs can be grafted onto other non-immunoglobulin scaffolds, such as Adnectins, Darpins, Adhirons, Alphabodies, Centyrins, Pronectins, Repebodies, Affimers, as well as Obodies and serve as binding agents for antigens.

This invention makes use of apheresis, generally. Apheresis of the blood including plasmapheresis (apheresis of the plasma), may be used to control levels of both the virus and gal-3, and more specifically biologically active galectin, in circulation. In this application, apheresis of whole blood, as well as separated plasma, may be used. Blood or plasma is lead through a fluid pathway and either intermixed with a virus binding agent which may be the same as or combined with a gal-3 binding agent. The binding agent is separated from the blood or plasma, or more preferably the blood or plasma is lead past a solid support on which is immobilized a binding agent which binds at least the virus and may bind gal-3. The virus, together with, if desired, gal-3 or other targets for selective withdrawal through apheresis are removed from the depleted blood or plasma, to which may be added conventional agents, is subsequently returned to the body, with a reduced viral load and, in preferred embodiments, a reduced level of gal-3. The term “apheresis” is used herein to refer to both apheresis where blood is passed by the binding agent, and plasmapheresis, where the blood cells are separated from plasma, and only plasma is passed by the binding agent(s).

This application is related to U.S. patent application Ser. No. 13/153,648, filed Jun. 6, 2011. That application in turn claims priority benefit to U.S. patent application Ser. No. 11/485,955, filed Jul. 6, 2006. The content of both these patent applications is expressly incorporated herein-by-reference. In U.S. patent application Ser. No. 13/153,648 (U.S. Patent Publication US-2011-0294755 A1) a method of treating cell proliferation conditions, inflammation and aggravated fibroses is disclosed which involves the administration of an agent that can bind circulating gal-3, such as modified citrus pectin, or MCP, a citrus pectin which has a reduced molecular weight of twenty thousand (20,000) Daltons or less, preferably ten thousand (10,000) Daltons or so. MCP is available commercially from EcoNugenics of Santa Rosa, Calif. as PectaSol-C, and is discussed in U.S. Pat. Nos. 6,274,566 and 6,462,029.

Independent of the binding agent selected, be it immobilized MCP, an antibody fragment bound to a magnet, an antibody bound to a specifically targetable support, the core invention disclosed herein calls for ex vivo treatment. No new agents, not previously established to be safe and effective, need be administered. Thus, particularly for individuals compromised by underlying or secondary conditions, this invention offers a method of treating COVID-10 infection, and the particularly damaging conditions associated therewith, that can be delivered immediately and safely.

Gal-3 is approximately 30 kDa and, like all galectins, contains a carbohydrate-recognition-binding domain (CRD) of about one hundred thirty (130) amino acids that enable the specific binding of β-galactosides. Gal-3 is encoded by a single gene, LGALS3, located on chromosome 14, locus q21-q22. This protein has been shown to be involved in a large number of biological processes. The list set forth herein is exemplary only as new situations and roles for gal-3 are continually being revealed. Among the biological processes at the cellular level that have been shown to be mediated, at least in part, by gal-3, are cell adhesion, cell migration, cell invasion, cell activation and chemoattraction, cell growth and differentiation, cell cycle, and apoptosis.

Given gal-3's broad biological functionality, it has been demonstrated to be involved in a large number of disease states or medical implications. Studies have also shown that the expression of gal-3 is implicated in a variety of processes associated with heart failure, including myofibroblast proliferation, fibrogenesis, tissue repair, inflammation, and ventricular and tissue remodeling. Elevated levels of gal-3 in the blood have been found to be significantly associated with increased morbidity and mortality. They have also been found to be significantly associated with higher risk of death in both acute decompensated heart failure and chronic heart failure populations. This is particularly pronounced in COVID-19 patients—the highest morbidity being positively strongly associated with high gal-3 levels.

As noted, elevated levels of circulating gal-3 are associated with, and apparently aggravate, a number of inflammatory conditions encountered in COVID-19 patients, including those contributing to heart, kidney, lung, and liver disease. Gal-3 is also associated with a fibrotic formation, particularly in response to organ damage, and associated with the “cytokine storm” phenomenon frequently observed in COVID-19 patients. Higher levels of circulating gal-3 are found to induce pathogenic fibroses in cardiovascular disease, gastroenterological disease, cardiovascular trauma, renal disease and tissue trauma, brain trauma, lung trauma, hepatic tissue trauma, tissue damage due to radiation therapy and diseases and conditions of connective tissue and skin such as systemic sclerosis. In the context of this invention—gal-3 mediates a number of reactions that result in activation or enhancement of various reactions (one example being the activation of TREM2 and another being the activation of TLR4.) Principally, however, beyond apheresis to selectively withdraw SARS-CoV-2 from the patient, reduction of gal-3 levels may reduce IL-6 and TNF Alpha activation, which may assist in limiting the “over reaction” to the viral infection—the creation of the storm that many patients cannot survive or survive but are plagued by long-term debilitating health consequences.

Particular attention has been focused on the development of critical kidney conditions aggravated or induced by COVID-19 infection. Although no standard treatments or practices have ben developed, acute kidney injury (AM) has been observed in up to twenty-five percent of patients critically ill with COVID-19 infection. Gabarre et al, Intensive Care Med., (2020). Early detection of kidney injury in these patients, and specific therapy which may be supported by reducing gal-3 levels may be critical. In a survey of over twenty-four thousand COVID-19 patients, AKI was predictive of higher severe infection and mortality rates. Shao et al, Pharmacological Research (pp. 1-12) (2020). In the absence of a new standard protocol for treatment of AM and chronic kidney infection related to COVID-19 infection, greater attention may be focused on controlling gal-3 levels together with existing treatments such as diuretics, acetylcysteine and sodium bicarbonate may be useful.

Plasmapheresis is a type of apheresis, where blood is diverted from the body through a needle or catheter to a separator which removes blood cells and returns them to the body, leaving a plasma. This type of technique has been used historically in the treatment of autoimmune diseases, where the antibodies at issue are removed by contacting the plasma with the ligands to which they bind. The plasma is then augmented as required, with anticoagulants, therapeutics and associated elements, and returned to the body. An early form of apparatus for plasmapheresis is set forth in U.S. Pat. No. 3,625,212, which describes measures to ensure return of treated plasma, as well as the separated blood cells, to the proper donor. U.S. Pat. No. 4,531,932 addresses plasmapheresis by centrifugation, the method used to separate out the red blood cells, on a rapid and near-continuous basis. U.S. Pat. Nos. 6,245,038 and 6,627,151 each describe a variety of methods of separating out plasma contents and returning the treated plasma to the patient after first removing red blood cells, in general, to reduce blood viscosity by removal of high molecular weight protein.

While the invention that is the subject of this application focuses on the reduction in SARS-CoV-2 viral particles and infection load, it may also be practiced, in a preferred embodiment, with apheresis to concomittantly reduce gal-3 levels. In this respect, the viral particles, and if desired gal-3, that are the principal “targets” of this invention, are “selectively withdrawn” as opposed to techniques that seek to separate out and rely on separation based on high molecular weight or viscosity. While apheresis and plasmapheresis techniques and devices—particularly using non-selective techniques to separate out a fraction of high molecular weight products are known—each of these patents is incorporated herein-by-reference for their disclosure of available plasmapheresis techniques and apparatus which may generally be employed in this invention. In a preferred embodiment of this invention, the apheresis device employed is that set forth in the referenced U.S. patent application Ser. No. 15/104302, currently pending. In this device, blood is withdrawn through a conduit that is connected to the patient's circulation, typically by a needle. The withdrawn blood may be separated in to cells and plasma if desired, but the emphasis in this invention is clearance of viral particles, which may lend itself to apheresis of the blood without separation. The blood or plasma continues, typically with the addition of an anticoagulant, to at least one column device where the patient's blood/plasma is exposed to the binding moiety, typically MCP or a SARS-CoV-2 specific antibody.

The binding moiety may be affixed to a support such as a magnetic particle that may be withdrawn from the body fluid after intermixing with a magnet or similar, such as easily bound beads or cartridges, or more typically is immobilized on the column or a filter provided with the column, and the blood/plasma flows through or past it, where it contacts the MCP or binding moiety, leaving the bound virus particle behind. The blood/plasma may be advanced by pumps where necessary. In another preferred embodiment, the blood or plasma circulates through the column device where the viral particles are bound—selective withdrawal of those particles is affected—as no general partitioning method is employed. Where desired, the blood or plasma circulates through another column device where gal-3 is selectively withdrawn. Since the same binding moiety—MCP target specific antibody—may be used, this suggests simply using the same column, or multiple similar columns. It is likely, however, that antibodies which bind more preferentially to SARS-CoV-2 than does MCP, may offer an advantage in terms of time and viral load reduction—the invention embraces both alternatives—if in addition to SARS-CoV-2 selective withdrawal, it is desired to reduce gal-3 levels, as discussed in detail in U.S. Pat. No. 8,764,395, then a longer column, or multiple columns, may be used. Such columns are available from Eliaz Therapeutics, Inc. of Santa Rosa Calif., packed with MCP or gal-3 antibody, under the mark XGAL3.

Advantageously, this treatment is combined with the administration of gal-3 blockers and inhibitors, and supportive treatments, such s those discussed above, including those such as disclosed in U.S. patent application Ser. No. 13/153,618. Although modified citrus pectin is a target inhibitor, other gal-3 inhibitors, such as other modified carbohydrates, including lactulosyl-l-leucine, Dermotte et al, Can. Res., 70 (19):476-88 (October 2010) as well as antibodies specific for gal-3, and other antagonists from very low molecular weight pectin weighing as low as 1 KD to higher molecular weight products such as GCS-100, Streetly et al, Blood, 115(19):3939-48 (published Feb. 26, 2010 as an abstract) may be used. GCS is a polysaccharide derived from MCP, as opposed to reduced MCP. A large variety of gal-3 binding antibodies are commercially available, from suppliers including abcam (ab2473), Novus Biologics (NB 100-91778) and Abgent (AJ13129). Other galectin-3 specific antibodies may be used. By removing large levels of plasma gal-3 from the blood, the disease, insult and injury due to inflammation or fibroses that is unfortunately commonly encountered in COVID-19 patients may be reduced, and the progression of the disease may be impeded. Similarly, conventional therapeutic treatments may be rendered more effective.

Typical circulating gal-3 level averages for a Caucasian adult range from 5 on up to about 20 ng/ml, with a value of 9-12 nanograms of gal-3 per milliliter of serum being a representative and reported value. Patients generally at risk, including those with advanced illnesses, exhibit levels, without treatment, that can be much higher than that patient's average or normal level. In accordance with the invention, individuals facing serious illness or continued COVID-19 related disability due to gal-3 mediated fibrosis, gal-3 mediated inflammation, and cancer growth, transformation and metastases associated with elevated gal-3 levels are treated by plasmapheresis to achieve a significant reduction in circulating gal-3 titer at the same time viral load is reduced.

By significant reduction in circulating gal-3 levels in the presence of COVID-19 infection, inflammation and/or fibrosis can be controlled. In general, a reduction of circulating gal-3 of at least ten percent (10%) is necessary to achieve significant progress in gal-3 mediated fibroses, and even more may be required in acute conditions involving inflammation, fibroses due to viral infection. In functional terms, the reduction of gal-3 should be sufficient to reduce or inhibit the impact of gal-3 levels on inflammation and fibroses in said patient. Reduction in circulating gal-3 of at least twenty percent (20%), and in some cases at least forty percent (40%) or even fifty percent (50%), may be required on a sustained basis. Severe situations, particularly critically ill COVID-19 patients experiencing their own cytokine storm may require reduction in circulating gal-3 levels in a mammalian patient of greater than fifty percent (50%) of that patient's circulating gal-3 titer, on up to seventy-five percent (75%) or even more. While some level of gal-3 in circulation is required for homeostasis, in acute situations, reductions at least by eighty percent (80%) of circulating gal-3, on up to near total removal of gal-3 from serum, may be called for, as that level is quickly replenished by the body.

The gal-3 levels in races other than Caucasians and subjects may vary, but regardless the target is to reduce gal-3 levels below the appropriate normal value. Viral particle reduction is the primary goal. If combined with gal-3 selective withdrawal, gal-3 target levels can vary based on the condition, age, gender, and other therapies involved. As a general matter, treatment of the patient according to this invention may begin with plasmapheresis designed to reduce the patient's gal-3 to a preselected value consistent with COVID-10 convalescence and recovery and to reduce infectious viral particles to levels that approximate newly infected, non-hospitalized patients. In some cases, it may be necessary to repeat or extend that treatment to achieve even greater reductions. Treatment can be repeated daily in the hospital setting and continued for prolonged periods of time in a hospital setting. Both viral particle count and Gal-3 can be easily monitored to determine the needed frequency. A single column or two parallel columns where one is regenerated while the other is working can be used. Such systems are commonly used in apheresis therapies.

This invention is straightforward in its application. It is recognizing how many different indications are served by this technology that is complex and startling. In the current invention, blood is removed from the patient according to well established protocols generally used for apheresis and plasmapheresis. For a general overview of this practice, see, Samuels et al, editors, Office Practice of Neurology, 1996. The removed blood may be treated, if desired, to remove blood cells from the plasma The blood can also be recycled and recirculated extra corporally, and filtered as needed, for a number of times (continuously) until the desired reduction in serum levels of SARS-CoV-2 and, if desired, gal-3 is achieved. Different serum levels can be targeted for different individuals. The blood cell-depleted plasma is then introduced to a chamber where viral particles and gal-3 are removed or inactivated by one or more binding antagonists. The binding agent may be modified to be complexed with an agent that is easily removed. In one embodiment, this is a magnetic particle. After providing for adequate circulation time, a magnetic field is applied to the fluid comprising the plasma and the MCP complex, and the bound gal-3 can be drawn off. In a preferred embodiment, the COVID-19 binder, and the gal-3 binder if not the same, are immobilized in a column through which the blood or plasma passes, contacting the immobilized binder. After passage, the column is discarded or regenerated.

Elevated circulating Gal-3 can impact a localized situation, such as localized inflammation or fibrosis in the lungs in a Covid-19 patient, and convert it into a larger, systemic problem. Thus, when gal-3 binds to components in the blood of a patient, which components also bind or include toxic agents, damaging ligands and the like, or similarly, when localized toxins are bound by gal-3, the damage potentially caused by these agents proximate to a localized injury or diseased tissue can become systemic. This is how the COVID-19 infection transforms into a deadly cytokine storm. Gal-3 is a generally adhesive molecule. Reducing elevated gal-3 levels below 15 or 12 ng/ml, by ten percent (10%) or more, will help to localize injury and damage, and maximize the benefit of unrelated therapeutic agents at the local injury or disease site.

In a preferred embodiment, the blood or serum, after having SARS-CoV-2 viral particles, and if desired, circulating gal-3 reduced or removed, by apheresis as described, is further treated before returning it to the patient's blood stream. Specifically, agents that may be more effective in the absence of, or in the presence of reduced levels of, gal-3 are specifically added. Some of these agents may be those used in the supportive treatments described above: antivirals, anti-inflammatories, immune based inhibitors, vitamin-based therapy and the like.

Agents not specific to viral infection may be provided as well. This includes a wide variety of active agents, but specifically includes agents such as chemotherapeutic drugs and therapeutic agents for the various conditions. For example, an anti-inflammatory will work better, cardiac medications, any drugs delivered to address an issue where COVID-19 infection and gal-3 are contributing factors, or prevent effective delivery to the target tissue, will be enhanced by this process. These agents will then have the opportunity to work under an environment of lower levels of gal-3. Even if just for a few hours, they can exhibit full biological activity. Once inflammation, for example, is reduced, naturally less gal-3 is being produced and expressed by the target tissue resulting in lower circulating gal-3 on a long-term basis. Thee agents can be administered orally, IV, IM, intranasal, in inhalation, when administered IV, the same IV access, post column, or different IV access can be used. They can be given during the apheresis, or shortly after.

While the present invention has been disclosed both generically, and with reference to specific alternatives, those alternatives are not intended to be limiting unless reflected in the claims set forth below. The invention is limited only by the provisions of the claims, and their equivalents, as would be recognized by one of skill in the art to which this application is directed.

What is claimed is:

1. A method of treating a patient suffering from COVID-19, comprising: conducting apheresis on the blood of said patient to reduce circulating levels of SARS-CoV-2 viral particles, wherein said apheresis comprises intimately contacting the viral particles in said blood with an agent that binds said viral particles, selectively withdrawing the bound viral particles from said blood, and returning said blood to said patient thereafter.

2. The method of claim 1 wherein said agent is modified citrus pectin of less sixty thousand Daltons molecular weight, an antibody which specifically binds to said viral particles, an antibody fragment which specifically binds to said viral particles, and mixtures thereof.

3. The method of claim 1, wherein said apheresis is conducted using the whole blood of said patient.

4. The method of claim 1, wherein the blood of said patient is treated to remove blood cells therefrom, creating a plasma which is then subjected to said apheresis, and wherein said blood cells are returned with said treated plasma to said patient.

5. The method of claim 1, wherein said apheresis further comprises selectively withdrawing gal-3 from said blood by intimately contacting said blood with an agent that binds gal-3 in said blood, selectively withdrawing bound gal-3 from said blood, and returning said blood to said patient.

6. The method of claim 5, wherein the agent that binds viral particles, and the agent that binds gal-3, both comprise modified citrus pectin.

7. The method of claim 1, wherein said binding agent is bound to a vehicle that facilitates said selective withdrawal, whereby said binding agent, following said intimate contact, is bound to said viral particles and separated from said blood by reason of said vehicle.

8. The method of claim 7, wherein said vehicle is a magnetic particle.

9. The method of claim 1, wherein said binding agent is caused to be intimately contacted by said viral particles by causing the patient's blood comprising the viral particles to pass through a module comprising an exposed surface on which said binding agent is immobilized, and passing said blood over said immobilized binding agent.

10. The method of claim 1, wherein said apheresis is repeated until said patient exhibits improved pathology such that said patient is unlikely to experience a cytokine storm due to COVID-19 infection.

11. The method of claim 3, wherein, upon return of said separated plasma to said mammal, the level of circulating gal-3 in said mammal is below 15 ng/ml.

12. The method of claim 1, wherein said method further comprises administering to said patient supportive treatment, comprising the administration of at least one of an antiviral agent, an anti-inflammatory agent, an immune reaction inhibitor, a vitamin and modified citrus pectin.
